SEQUENCE LISTING (6 sequences)

SEQ ID NO: 1 - DNA, 1715 bp, Homo sapiens
  CDS (150)...(1484)
gaaccttcctgccgtcgcgtttgcacctcgctgctccagcctctggggcgcattccaaccttccagcctgcgacctgcggagaaaaaaaattacttattttcttgccccatacataccttgaggcgagcaaaaaaattaaattttaaccatgagggaaatcgtgcacatccaggctggtcagtgtggcaaccagatcggtgccaagttctgggaggtgatcagtgatgaacatggcatcgaccccaccggcacctaccacggggacagcgacctgcagctggaccgcatctctgtgtactacaatgaagccacaggtggcaaatatgttcctcgtgccatcctggtggatctagaacctgggaccatggactctgttcgctcaggtccttttggccagatctttagaccagacaactttgtatttggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaaaaaaaaaaaaaaaaaaaaaaaaaaaaaaaaaaaaaaaaaaaaaaaaaaaa

SEQ ID NO: 2 - protein, 444 aa, Homo sapiens
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 3 - DNA, 21 bp, Artificial Sequence
  Oligonucleotide primer
ctccgcaagttggcagtcaac

SEQ ID NO: 4 - DNA, 21 bp, Artificial Sequence
  Oligonucleotide primer
ggggatccattccacaaagta

SEQ ID NO: 5 - DNA, 19 bp, Artificial Sequence
  Oligonucleotide primer
ttggcagtcaacatggtcc

SEQ ID NO: 6 - DNA, 26 bp, Artificial Sequence
  Oligonucleotide primer
cgttaagcatctgctcatcgacctcc

(19) United States
(12) Patent Application Publication
(10) Pub. No.: US 2007/0190544 A1
(43) Pub. Date: Aug. 16, 2007

(54) Methods of screening for resistance to microtuble-targeting drugs

(75) Inventors: Paraskevi Giannakakou, Washington Township, NJ (US); Wei Zhou, Lilburn, GA (US); Yuefang Wang, Lawrenceville, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Emory University

(21) Appl. No.: 11/589,534

(22) Filed: Oct. 30, 2006

Related U.S. Application Data
(60) Provisional application No. 60/731,379, filed on Oct. 28, 2005.

(51) Int. Cl. C12Q 1/68

(52) U.S. Cl. 435/6

(57) ABSTRACT

The invention relates to methods for determining resistance or responsivity to microtubule-targeting drug treatment in cancer patients. The methods comprise obtaining a tumor cell sample from a cancer patient and analyzing DNA in the tumor cell sample to determine the presence or absence of a loss of heterozygosity (LOH) at the M40 β-tubulin gene locus within chromosomal locus 6p25, where determining LOH comprises screening for at least one mutation in the M40 β-tubulin gene that affects the binding of a microtubule-targeting drug to β-tubulin. In such methods, the presence of LOH is indicative of microtubule-targeting drug resistance in the cancer patient or of a decreased likelihood that the cancer patient will respond to therapy with a microtubule-targeting drug.

### CROSS-REFERENCE TO RELATED APPLICATIONS

This application claims the benefit of U.S. Provisional Patent Application No. 60/731,379, filed Oct. 28, 2005, which is hereby incorporated by reference in its entirety.

### FEDERALLY SPONSORED RESEARCH OR DEVELOPMENT

This invention was made with government support under grant numbers NCI 1R01 CA100202-01 and NCI Supplement to R01 CA86335 awarded by the National Cancer Institute. The United States government has certain rights in the invention.

### FIELD OF THE INVENTION

The invention relates to methods for determining resistance or predicting responsivity to microtubule-targeting drug treatment in cancer patients.

### BACKGROUND

Microtubules are major dynamic structural components in cells. They are important for development and maintenance of cell shape, cell division, cell signaling, and cell movement. Microtubules are cytoskeletal polymers built by the self-association of α and β-tubulin dimers, existing in a constant dynamic equilibrium between their polymerized microtubule form and soluble α and β-tubulin dimer forms. Drugs that target tubulin or microtubules are one of the most effective classes of anticancer agents. These drugs bind to different sites on the tubulin dimer and within the microtubule, exerting varying effects on microtubule dynamics. However, they all block cells in mitosis at the metaphase/anaphase transition and induce cell death (Jordan et al. (1993) Proc. Natl. Acad. Sci. USA 90:9552-9556). Among all the microtubule-targeting drugs, the taxanes (e.g., Taxol®), are arguably the most effective anticancer agents used to date in clinical oncology due to their remarkable activity in a broad range of malignancies.

The epothilones, another group of microtubule-targeting drugs, are novel microtubule-stabilizing products of soil bacteria origin that compete with Taxol® for the same binding site on β-tubulin. In an effort to better understand how the epothilones interact with microtubules Giannakakou et al. isolated two epothilone-resistant human ovarian cancer cell lines, 1A9-A8 and 1A9-B10, that were selected with epothilone A and B respectively ((2000) Proc. Natl. Acad. Sci. USA 97:2904-2909). These epothilone-resistant sublines exhibit impaired epothilone- and Taxol®-driven tubulin polymerization, caused by the following acquired β-tubulin mutations in each clone: β274 (Thr to Ile) in 1A9-A8 cells and β282 (Arg to Gln) in 1A9-B10 (Giannakakou et al. (2000) Proc. Natl. Acad. Sci. USA 97:2904-2909). These mutations are located at the Taxol®-binding site in the atomic model of αβ-tubulin (Nettles et al. (2004) Science 305:866-869).

Despite the clinical success of microtubule-targeting drugs and other chemotherapeutic agents, the emergence of drug-resistant tumor cells limits the ability of these compounds to cure disease. In fact, acquired drug resistance is the primary reason for chemotherapy failure in patients that may have initially responded to the treatment. In the case of the microtubule-targeting drug Taxol®, several mechanisms of resistance have been described. With the exception of P-glycoprotein (Pgp)-mediated multi-drug resistance (MDR) (Reinecke et al. (2000) Cancer Invest. 18:614-625; Horwitz et al. (1993) J. Natl. Cancer Inst. Monogr. 15:55-61), all these mechanisms involve alterations in tubulin. Such alterations include: (1) altered expression of β-tubulin isotypes in Taxol®-resistant cells and Taxol®-resistant ovarian tumors (Haber et al. (1995) J. Biol. Chem. 270:31269-31275; Jaffrezou et al. (1995) Oncol. Res. 7:517-27; Kavallaris et al. (2001) Cancer Research 61:5803-5809); (2) increased microtubule-dynamics in Taxol®-resistant cancer cells (Goncalves et al. (2001) Proc. Natl. Acad. Sci. USA 98:11737-11742), and (3) the presence of β-tubulin mutations in Taxol®-resistant cells (Giannakakou et al. (1997) J. Biol. Chem. 272:17118-17125; Gonzalez-Garay et al. (1999) J. Biol. Chem. 274:23875-23882).

Development of drug resistance to cancer chemotherapeutic agents (e.g., microtubule-targeting drugs such as Taxol®) via gene mutations or other alterations of the cellular targets of these drugs is a major obstacle in clinical oncology. Accordingly, a need exists for improved methods to analyze the status of target genes.

### SUMMARY OF THE INVENTION

Methods for determining microtubule-targeting drug resistance in a cancer patient are provided. The methods comprise obtaining a tumor cell sample from a cancer patient and analyzing DNA in the tumor cell sample to determine the presence or absence of a loss of heterozygosity (LOH) at the M40 β-tubulin gene locus within chromosomal locus 6p25, where determining LOH comprises screening for at least one mutation in the M40 β-tubulin gene that affects the binding of a microtubule-targeting drug to β-tubulin. In such methods, the presence of LOH is indicative of microtubule-targeting drug resistance in the cancer patient.

Methods are also provided for predicting the likelihood that a cancer patient will respond to therapy with a microtubule-targeting drug. The methods comprise obtaining a tumor cell sample from a cancer patient and analyzing DNA in the tumor cell sample to determine the presence or absence of LOH at the M40 β-tubulin gene locus within chromosomal locus 6p25, where determining LOH comprises screening for at least one mutation in the M40 β-tubulin gene that affects the binding of a microtubule-targeting drug to β-tubulin. In such methods, the presence of LOH is indicative of a decreased likelihood that the cancer patient will respond to therapy with a microtubule-targeting drug.

In certain embodiments of the methods of the present invention, the microtubule-targeting drug may be a microtubule-stabilizing drug, such as a taxane or epothilone, or a microtubule-destabilizing drug, such as vincristine. In further embodiments, the mutation in the M40 β-tubulin gene results in an amino acid substitution in β-tubulin at amino acid positions 26, 172, 198, 231, 240, 270, 274, 282, 292, 350, or 364, particularly where the amino acid substitution is Asp26Glu, Ser172Ala, Glu198Gly, Ala231Thr, Leu240Ile, Phe270Val, Thr274Ile, Thr274Pro, Arg282Gln, Gln292Glu, Lys350Asn, or Ala364Thr. In further embodiments, the tumor cell sample comprises tumor cells of a type selected from the group consisting of breast cancer, ovarian cancer, colon cancer, prostate cancer, liver cancer, lung cancer, gastric cancer, esophageal cancer, urinary bladder cancer, melanoma, leukemia, and lymphoma.

### BRIEF DESCRIPTION OF THE DRAWINGS

FIG. 1 illustrates that 1A9/A8 and 1A9/A8E cells exhibit impaired in vivo drug-induced tubulin polymerization compared with their parental 1A9 cells. Drug-sensitive parental 1A9 (top panel) and the Epo A-resistant clones, 1A9/A8 and 1A9/A8E (lower panel), were treated for 5 hours with or without (0) various concentrations of Epo A as indicated. After cell lysis, the polymerized (P) and the soluble (S) protein fractions were separated by centrifugation, resolved by SDS/PAGE, and immunoblotted with an antibody against alpha-tubulin. The percent of polymerized tubulin (% P) was determined by dividing the densitometric value of polymerized tubulin by the total tubulin content (the sum of P plus S). The results shown are from a representative experiment of four independent observations.

FIG. 2 shows impaired Epothilone-induced G2/M arrest in the 1A9-Epo^{R} cells. Cell cycle analysis by flow cytometry was performed in the parental 1A9 and the 1A9-Epo^{R} clones, following overnight treatment with Epo A or Vincristine (VCR) as indicated. The parental 1A9 cells readily arrested in G2/M, after treatment with either microtubule-stabilizing or destabilizing agents. The early-step isolate 1A9-A8E was partially arrested in G2/M following Epo A treatment, while the late-step isolate 1A9-A8 failed to arrest in mitosis after treatment with Epo A, even at the highest concentration. Both Epo-resistant clones were arrested in G2/M after treatment with 10 nM Vincristine. 10,000 events were recorded for each condition, the histogram is representative of three independent experiments.

FIG. 3 shows that M40, the major isotype of β-tubulin in human cells, is located on 6p25. PCR amplification from genomic DNA isolated from the BAC clones RP11-527J5 BAC (located at 6p21.3) and RP11-506K6 BAC (located at 6p25), using primers specific for M40 and β9 tubulin isotypes. The primers were designed from intron 3 to the 3′-UTR region of each gene, thereby amplifying the entire exon 4. Upon bacterial expansion of the two BACs, five different clones were picked to ensure that the BACs were uniform and homogeneous. As a positive control, genomic DNA from 1A9 cells was used.

FIG. 4 illustrates the methodology used for SNP marker analysis of 1A9 and 1A9-resistant cells. Left Panel. Diagram of chromosome 6p displaying the location of the SNP markers within the 9.5 Mb contig NT_{—}003488. The β-tubulin gene M40 is highlighted within the BAC clone RP11-506k6 located within this contig at 6p25. The location of the four informative SNP markers is displayed. Right Panel. Table showing the corresponding SNP nucleotides by DNA sequencing analysis in the parental 1A9 cells, and the four drug-resistant clones, as indicated.

### DETAILED DESCRIPTION

The present invention relates to methods for determining microtubule-targeting drug resistance or predicting responsivity to microtubule-targeting drug treatment in cancer patients. The methods comprise obtaining a tumor cell sample from a cancer patient and analyzing DNA in the tumor cell sample to determine the presence or absence of a loss of heterozygosity (LOH) at the M40 β-tubulin gene locus within chromosomal locus 6p25, where determining LOH comprises screening for at least one mutation in the M40 β-tubulin gene (SEQ ID NO:1) that affects the binding of a microtubule-targeting drug to β-tubulin (SEQ ID NO:2). In such methods, the presence of LOH is indicative of microtubule-targeting drug resistance in the cancer patient or of a decreased likelihood that the cancer patient will respond to therapy with a microtubule-targeting drug. In specific embodiments, the microtubule-targeting drug is a microtubule-stabilizing drug such as a taxane or epothilone, including Taxol® (paclitaxel), epothilone A, epothilone B, or analogs, derivatives, or prodrugs thereof. In further embodiments, the microtubule-targeting drug is a microtubule-destabilizing drug such as a Vinca alkaloid, cryptophycin, or colchicines, including vinblastine and vincristine. In particular embodiments, the M40 β-tubulin gene mutation results in an amino acid substitution in ,tubulin at amino acid positions 26, 172, 198, 231, 240, 270, 274, 282, 292, 350, or 364, particularly where the amino acid substitution is Asp26Glu, Ser172Ala, Glu198Gly, Ala231Thr, Leu240Ile, Phe270Val, Thr274Ile, Thr274Pro, Arg282Gln, Gln292Glu, Lys350Asn, or Ala364Thr.

Microtubule-targeting drugs form one of the most effective classes of anticancer agents, and the size of this class continues to expand. Microtubule-targeting agents are divided into two main groups according to their effects on microtubule polymer mass. The first group is composed of microtubule-destabilizing drugs that bind preferentially to tubulin dimers and inhibit tubulin assembly. These include the Vinca alkaloids, cryptophycins, and colchicines, as described more fully herein below. The second group is composed of microtubule-stabilizing drugs that bind preferentially to the microtubule polymer, enhance tubulin polymerization and include the taxanes (such as Taxol® and Taxotere), epothilones, eleutherobins, laulimalide, and discodermolide. Although all these drugs bind to distinct sites on the microtubule or the tubulin dimer, they all affect microtubule dynamics, block mitosis at the metaphase/anaphase transition, and consequently induce cell death (for review see Jordan (2002) Curr. Med. Chem. 2:1-17). Since cancer cells are more dynamic and divide at much higher rates than normal cells, microtubule-targeting drugs are most toxic to cancer cells. The emergence of drug resistant tumor cells has limited the ability of drugs such as Taxol® to cure disease.

As described above, the methods disclosed herein find use in determining microtubule-targeting drug resistance or predicting responsivity to microtubule-targeting drug treatment in cancer patients. As used herein, “microtubule-targeting drug resistance” refers to a state of insensitivity or decreased sensitivity of cancer cells to drugs that would ordinarily cause cell death. This resistance can be either intrinsic or acquired. Intrinsic resistance may be defined as a state of insensitivity to initial therapy in response to a drug or combination of drugs. On the other hand, acquired drug resistance may be defined as a state whereby a population of cancer cells that were initially sensitive to a drug undergoes a change towards insensitivity. Acquired drug resistance is the most common reason for the failure of drug treatment in cancer patients with initially sensitive tumors, and as such, is presently responsible for the majority of deaths from cancer. By “predicting responsivity to microtubule-targeting drug treatment” is intended assessing the likelihood that a patient will respond to therapy with a microtubule-targeting drug (i.e., will not exhibit microtubule-targeting drug resistance).

As described above, the methods of the present invention comprise determining the presence or absence of a LOH at the M40 β-tubulin gene locus within chromosomal locus 6p25, where determining LOH comprises screening for at least one mutation in the M40 β-tubulin gene that affects the binding of a microtubule-targeting drug to β-tubulin. As used herein, “loss of heterozygosity” or “LOH” refers to loss of one of the two alleles at one or more loci in a cell line or cancer cell population due to chromosome loss, deletion, or mitotic crossing-over. If one of a pair of heterozygous alleles is lost due to a deletion of DNA from one of the paired chromosomes, only the remaining allele will be expressed and the affected cells are functionally homozygous. Following this loss of an allele from a heterozygous cell, the protein or gene product thereafter expressed will be homogeneous because all of the protein will be encoded by the single remaining allele. The cell becomes effectively homozygous at the gene locus where the deletion occurred. Almost all, if not all, varieties of cancer cells undergo LOH at some chromosomal regions.

Techniques for analyzing LOH at any particular locus are well known (See, e.g., Deng et al. (1994) Cancer Res. 54:499-505; Matsumoto et al. (1997) Genes, Chromosomes & Cancer 20:268-274). Within the methods of the present invention, LOH at the M40 β-tubulin gene locus within chromosomal locus 6p25 may be analyzed by any of these well known techniques or as described in the Examples provided herein. In the typical case, the target cancer cells to be analyzed will be substantially isolated from other cell populations. Chromosomal DNA may be isolated from the target cells by any of a number of techniques that are well known in the art. In some cases, the subsequent analysis of LOH will not require that the DNA be completely or even substantially removed from other cell components. Typically, the polymorphic regions of the chromosomes to be analyzed are amplified by PCR using appropriate primers. Many polymorphic markers for the M40 β-tubulin gene locus within chromosomal locus 6p25 are well known and can be found at the web site for the National Center for Biotechnology Information of the National Library of Medicine of the National Institutes of Health (http://www.ncbi.nlm.nih.gov). Primers that are particularly useful for amplifying the M40 β-tubulin gene locus within chromosomal locus 6p25 include CTCCGCAAGTTGGCAGTCAAC (SEQ ID NO:3), GGGGATCCATTCCACAAAGTA (SEQ ID NO:4), TTGGCAGTCAACATGGTCC (SEQ ID NO:5), and CGTTAAGCATCTGCTCATCGACCTCC (SEQ ID NO:6) (See Table 4 in the Examples section below).

The amplified regions from each heterozygous allele will differ in some detectable property, for example, size or restriction sites, such that two distinctive patterns are produced from heterozygous loci. When a deletion of one of the alleles occurs, only one of the patterns can be detected, hence there is LOH in that locus. In most cases, the LOH will not appear as a complete loss of the pattern from the deleted allele (because the analysis will be carried out on a population of cells, not all of whose chromosomes necessarily exhibit an allelic deletion), but as a decrease in the intensity of the signal from one allele. The intensities of the allelic signals for the target tumor cell sample can be measured against the allelic signals from a control cell sample for a comparison. The control cell sample for LOH analysis will be a non-tumor cell sample from the same individual from whom the target tumor cell sample is obtained. Exemplary control cell samples can be skin, lymph node or blood cell samples. One of ordinary skill in the art is competent to select other appropriate control cell samples for use in determining LOH within the methods of the present invention.

As described above, the methods of the present invention comprise determining the presence or absence of LOH at the M40 β-tubulin gene locus within chromosomal locus 6p25, where determining LOH comprises screening for at least one mutation in the M40 β-tubulin gene that affects the binding of a microtubule-targeting drug to β-tubulin. One of skill in the art will readily be able to identify mutations in the M40 β-tubulin gene that affect the binding of microtubule-targeting drugs to β-tubulin. Numerous studies have been performed to identify acquired tubulin mutations in human cancer cell lines which confer resistance to either microtubule-stabilizing or microtubule-destabilizing drugs, as described more fully below. Furthermore, structural analyses of protein-ligand complexes and computationally refined electron crystallographic structures provide guidance to one of skill in the art regarding which mutations in the M40 β-tubulin gene would be expected to affect the binding of a microtubule-targeting drug to β-tubulin.

Currently, there are five known drug binding sites on β-tubulin. They have names assigned depending on which drug was originally found to bind the site. The taxane binding site on β-tubulin is shared by drugs that stabilize microtubules and bind preferentially at the microtubule polymer (Rao et al. (1992) J. Natl. Cancer Inst. 84:785-788; Rao et al. (1994) J. Biol. Chem. 269:3132-3134; Rao et al. (1995) J. Biol. Chem. 270: 20235-20238; Rao et al. (1999) J. Biol. Chem. 274:37990-37994; Nogales et al. (1998) Nature 391:199-203). The prototype of this class of drugs is Taxol®, but newer members include the epothilones, discodermolide, eleutherobin, and the sarcodictyins (Schiff et al. (1979) Nature 391:199-203; Bollag et al. (1995) Cancer Res. 55:2325-2333; ter Haar et al. (1996) Biochemistry 35:243-250; Long et al. (1998) Cancer Res. 58:1111-1115). Three of the other binding sites are shared by drugs that bind preferentially to unpolymerized tubulin, inhibiting tubulin assembly. These destabilizing agents either form covalent crosslinks to tubulin cysteine residues such as Cys-β239, such as the small molecules 2,4-dichlorobenzyl thiocyanate and T138067; bind tubulin at the colchicine site such as the combretastatins, curacins, 2-methoxyestradiol, and the podophylotoxins; bind tubulin at the Vinca domain, such as maytansin, rhizoxin; or locate in alpha-tubulin as do the hemiasterlins, which also bind at the Vinca domain, and perhaps the cryptophycins (Bai et al. (1989) Biochemistry 28:5606-5612; Shan et al. (1999) PNAS USA 96:5686-5691; Pettit et al. (1988) J. Nat. Prod. 51:517-527; Blokhin et al. (1995) Mol. Pharmacol. 48:523-531; D'Amato et al. (1994) PNAS USA 91:3964-3968; Wilson et al. (1970) Biochemistry 9:4999-5007; Lin et al. (1981) Res. Commun. Chem. Pathol. Pharmacol. 31:443-451; Bai et al. (1990) J. Biol. Chem. 265:17141-17149; Nunes et al. (2002) Eur. J. Cancer 38:S119; Bai et al. (1999) Biochemistry 38:14302-14310; Hamel et al. (2002) Curr. Med. Chem. Anti-Canc. Agents 2:19-53). The fifth binding site, on tubulin, has been recently identified as the location where the microtubule-stabilizing drug laulimalide binds (Pryor et al. (2002) Biochemistry 41:9109-9115). Recently, a microtubule-stabilizing natural product derived from a New Zealand marine sponge, peluroside, was also found to compete with laulimalide for this site (Pineda et al. (2004) Bioorg. Med. Chem. Lett. 14:4825-4829).

As described above, a number of acquired tubulin mutations in human cancer cell lines which confer resistance to either microtubule-stabilizing or microtubule-destabilizing drugs are known in the art. For example, the taxanes, Taxol® and its semisynthetic analog Taxotere, bind preferentially and with high affinity to the β-subunit of the tubulin dimer along the entire length of the microtubule. Electron crystallographic studies on Taxol® complexed with tubulin have allowed the precise identification of the amino acids which comprise the taxane binding pocket on β-tubulin and have revealed the location of the site at the inside lumen of the microtubule (Nogales et al. (1998) Nature 391:199-203; Nogales et al. (1999) Cell 96:79-88). Sequence alterations of single amino acids at the taxane binding site are known to have a significant impact on the drug's ability to bind tubulin. For example, two distinct β-tubulin mutations in two human ovarian cancer clones have been selected independently with Taxol® (Phe270Val and Ala364Thr) (Giannakakou et al. (1997) J. Biol. Chem. 272:17118-17125). As a result, impaired Taxol®-induced tubulin polymerization was observed in the two clones, which were found to exhibit a 30-fold resistance to Taxol® (fold resistance is calculated as the ratio of the drug's IC 50 against the resistant cell line over the drug's IC 50 against the respective parental cell line). Subsequent reports have described other acquired β-tubulin mutations in human breast cancer cells and human epidermoid cancer cells following Taxol® selections (Glu198Gly and Asp26Glu, respectively) (Wiesen et al. (2002) Proc. Amer. Assoc. Cancer Res. 43:788; Hari et al. (2006) Mol. Cancer. Ther. 5:270-278). The Glu198Gly mutation, located near the α/β interphase, confers 17-fold resistance to Taxol® and some cross resistance to Taxotere and the epothilones, while the Asp26Glu mutation at the N-terminus of β-tubulin (which forms part of the taxane binding pocket Nogales et al. (1998) Nature 391:199-203), and confers an 18-fold resistance to Taxol® with minimal cross-resistance to epothilone B and MAC-231, yet 10-fold cross resistance to Taxotere.

Detailed structural analyses using the protein-ligand complexes of Taxol® (paclitaxel or PTX) (Snyder et al. (2001) PNAS USA 98:5312-5316; Lowe et al. (2001) J. Mol. Biol. 313:1045-1057) as well as epothilone A (EpoA) (Nettles et al. (2004) Science 305:866-869) from computationally refined electron crystallographic structures have been performed and provide guidance to one of skill in the art regarding mutations in the M40 β-tubulin gene that would be expected to affect the binding of a microtubule-targeting drug to β-tubulin. For example, of four mutations in β-tubulin that arose in response to selection with Taxol® (PTX or paclitaxel), three of were found to be clustered in the taxane binding site, in direct contact with bound drug. The first two mutations (βPhe270Val and βAla364Thr) were observed in 1A9 ovarian cancer cells (Giannakakou et al. (1997) J. Biol. Chem. 272:17118-17125) and are closely associated with Taxol® in the microtubule protein. Phe270 is in van der Waals contact with Taxol®'s C-3′ phenyl group, while Ala364 resides in a five-residue hydrophobic cluster at the bottom of the tubulin binding pocket housing the Ligand (side-chains immediately associated with Taxol® include Phe270, Pro272, Pro358 and Leu 361). The mutation of β364 from a nonpolar alanine to a polar threonine can be expected to cause reorganization of this cluster, with consequences for binding affinity with the ligand.

The Asp26Glu change in KB-3-1 epidermal cells observed in response to Taxol®-selection is likewise accompanied by direct contact between protein and Taxol® (Hari et al. (2006) Mol. Cancer. Ther. 5:270-278). In particular, the CH₂ of the Asp side chain is at the van der Waals boundary with respect to two CH centers of the phenyl ring of Taxol®'s C-3′ benzamido group. The same methylene abuts one methyl group of the t-butyl group of taxotere docked in the same site. The steric resistance between drugs and protein side chain permits a hydrogen bond between the Asp26 carboxylate and taxotere's NH, but only a longer range electrostatic interaction for Taxol® (Hari et al. (2006) Mol. Cancer. Ther. 5:270-278). Replacement of Asp26 with Glu causes 18-fold resistance to Taxol®, but also results in a decrease in microtubule stability, likely responsible for the drug-dependent nature of these cells. Importantly, this mutation creates only 3- to 5-fold resistance to taxotere, as well as to a furan-containing analog, MAC-321. One of the outcomes of extending the Asp chain by an extra methylene unit (CH₂) in Glu, is to bring the negatively charged carboxylate functionality (i.e., CO₂—) in closer contact with the NHCO centers of the ligands. Taxotere and MAC-321 persist in a productive hydrogen bond with the lengthened Glu side chain, but severe steric interactions of the same Glu conformation with Taxol® appear to force this drug up and out of the binding pocket. As a result, it has been proposed that in the case of Taxol® an alternative Glu side chain conformation is adopted; one that does not contribute to ligand binding (Hari et al. (2006) Mol. Cancer. Ther. 5:270-278).

In addition to mutations affecting the binding of taxanes to β-tubulin, other mutations in the M40 β-tubulin gene known to affect the binding of epothilones to β-tubulin are also known in the art. Although epothilones are structurally distinct from Taxol®, they compete for the same binding site and exert similar microtubule-stabilizing activity. Two epothilone-resistant human ovarian cancer cell lines selected with epothilone A and B have been shown to exhibit impaired epothilone- and Taxol®-driven tubulin polymerization caused by individual acquired β-tubulin mutations (Thr274Ile and Arg282Gln) (Giannakakou et al. (2000) PNAS USA 97:2904-2909). Both of these mutations are located near the Taxol®-binding site in atomic models of αβ-tubulin, explaining why these cells are also cross-resistant to Taxol® (7-10 fold). Interestingly, a different alteration in residue 274 (Thr274Pro) has been identified in an epothilone A-selected human epidermoid carcinoma cell line, which conferred 45-fold resistance to epothilone A, 8-fold resistance to epothilone B and significant cross-resistance to Taxol® (96-fold) (Mehdi et al. (2001) Proc. Amer. Assoc. Cancer Res. 42:920). The fact that two different human cancer cell lines, selected by two different research groups, acquire mutations at the same residue in response to epothilone selection suggests that this specific residue is very important for epothilone binding to tubulin and that it may prove to be a “hot spot” for acquired tubulin mutations following epothilone treatment.

Additional studies have reported the presence of acquired β-tubulin mutations in non-small cell lung cancer (NSCLC), Hela, and human leukemia cells (He et al. (2001) Mol. Cancer Ther. 1:3-10; Verrills et al. (2003) Chem. Biol. 10:597-607). A Gln292Glu mutation has been identified conferring 70-95 fold resistance to the epothilones and significant cross-resistance to Taxol® (22-fold) and Taxotere (13-fold) (He et al. (2001) Mol. Cancer Ther. 1:3-10). Selection of cancer cells with increasing concentrations of the epothilone B analog, desoxyepothilone B (dEpoB) has also identified mutations Ala231Thr and Gln292Glu (Verrills et al. (2003) Chem. Biol. 10:597-607).

As with Taxol®, one of skill in the art will readily appreciate the structural relationship between mutations in the M40 β-tubulin gene that impact epothilone binding sites in β-tubulin. For example, a recent electron crystallography model of the binding of EpoA to β-tubulin illustrates that Thr274 and Arg282 of tubulin are jointly engaged in a network of hydrogen bonds with the oxygens at C-3, C-5 and C-7 on the epothilone A ligand (Nettles et al. (2004) Science 305:866-869). The mutation replacing Thr with Ile on residue β274 not only eliminates the βThr274-mediated tubulin-Epo interaction, but obviates the β274Thr-β282Arg interaction while impacting on the β282Arg-mediated tubulin-Epo contacts as well. The close association of Thr274 and Arg282 implies that mutation of Arg282 would be predicted to disengage Thr274 in a reciprocal manner; a prediction supported by the observation that in EpoB-selected 1A9/B10 ovarian cancer cells, a mutation of βArg282Gln confers 24-53 fold resistance to that ligand, and 57-74 fold cross-resistance to EpoA. This amino acid change not only shrinks the side chain by two heavy atoms (i.e., by —CH₂—CH₂—), eliminating a direct hydrogen bond, but also removes the positive charge and damps any longer-range electrostatic stabilization as well.

In another example of the structural relationship between mutations in the M40 β-tubulin gene that impact epothilone binding sites in β-tubulin, the alanine at amino acid position is not in direct contact with the ligand, but lies on helix H7, deep in the β-tubulin binding pocket at a distance of 7-8 Å from the ligand and surrounded largely by hydrophobic residues (i.e., Val23, Leu228, Gly235, Phe270 and Leu273). Helix H7 is regarded as being central to the conformation of tubulin and is one of the structural elements that forms one wall of the hydrophobic taxane pocket, as illustrated by the subset of residues from His227 to Gly235 that bracket Ala231 (Amos & Lowe (1999) Chem. Biol. 6:R65-69). A change in this residue may perturb the normal interaction of His227 with dEpoB. One edge of the Ala231 cluster is populated by His227, which provides an anchor to the epothilone thiazole side chain (Nettles et al. (2004) Science 305:866-869). The replacement of alanine with threonine is predicted to result in two alterations of the environment around the ligand. First, addition of a polar OH group to the CH₃ of Ala to give the CH₂OH in Ser can be expected to perturb both the small pool of water between the ligand and the tubulin protein, thus influencing the shape of the hydrophobic cluster. Second, the somewhat extended serine side chain is in a position to compete with the epothilone thiazole moiety for hydrogen bonding to His227. Both these actions can be seen as deleterious for dEpoB binding and therefore responsible for the considerable degree of the resistance observed.

Other mutations in the M40 β-tubulin gene that affect the binding of microtubule-targeting drugs are also known in the art. For example, antimitotic drugs that inhibit the binding of colchicine to tubulin appear to bind at a common site called the colchicine site. Although the prototype of this class is colchicine, it includes podophylotoxin, 2-methoxyestradiol (2ME2) and indanocine, among others. Human T-lymphoblatoid CEM cells selected with indanocine exhibited a drug-resistant phenotype due to an acquired Lys350Asn β-tubulin mutation (Hua et al. (2001) Cancer Res. 61:7248-7254; Ravelli et al. (2004) Nature 428:198-202). In contrast to the colchicine-binding site, antitubulin compounds that inhibit the binding of radiolabeled vinblastine and vincristine to tubulin share a common binding site described as the “Vinca domain”. The prototypes of this class are the Vinca alkaloids, vinblastine and vincristine (Johnson et al. (1960) Cancer Res. 20:1016-1022; Cutts et al. (1960) Cancer Res. 20:1023-1031). Several other naturally occurring microtubule-interfering compounds have been identified that bind β-tubulin at the Vinca domain, including the hemasterlins, halichondrins, spongistatin, dolastatins, and cryptophycins (isolated from the blue-green algae Nostoc sp.) (for review see Zhou et al. (2005) Curr. Med. Chem. Anti-Cancer Agents 2:55-70). Acquired tubulin mutations in human cancer cells for the Vinca domain have been reported in response to selections with vincristine (Leu240Ile) and the hemiasterlin analog HTI-286 (Ser172Ala) (Kavallaris et al. (2001) Cancer Res. 61:5803-5809; Poruchynsky et al. (2004) Biochemistry 43:13944-13954; Loganzo (2004) Mol. Cancer Ther. 3:1319-1327). One of skill in the art will readily appreciate the structural relationship between the mutations described above and the binding sites of their respective drugs in β-tubulin (see, e.g., Sawada et al. (1993) Biochem. Pharmacol. 45:1387-1394; Rai et al. (1996) J. Biol. Chem. 271:14707-14711; Haber et al. (1989) Cancer Res. 49:5281-5287; Bai et al. (2000) J. Biol. Chem. 275:40443-40452; Gupta et al. (2003) Mol. Cell Biochem. 253:41-47).

Accordingly, in specific embodiments, the microtubule-targeting drug encompassed by the methods of the present invention is a microtubule-stabilizing drug such as a taxane or epothilone, including Taxol® (paclitaxel), epothilone A, epothilone B, or analogs, derivatives, or prodrugs thereof. In a further specific embodiment, the microtubule-targeting drug encompassed by the methods of the present invention is a microtubule-destabilizing drug such as a Vinca alkaloid, cryptophycin, or colchicines, including vinblastine and vincristine. In further particular embodiments, the M40 β-tubulin gene mutation within the methods of the present invention results in an amino acid substitution in β-tubulin at amino acid positions 26, 172, 198, 231, 240, 270, 274, 282, 292, 350, or 364, particularly where the amino acid substitution is Asp26Glu, Ser172Ala, Glu198Gly, Ala231Thr, Leu240Ile, Phe270Val, Thr274Ile, Thr274Pro, Arg282Gln, Gln292Glu, Lys350Asn, or Ala364Thr.

The methods of the present invention find use in determining microtubule-targeting drug resistance or predicting the responsivity to microtubule-targeting drug treatment for cancer patients diagnosed with any cancer type. In particular embodiments of the present invention, the methods comprise obtaining samples of tumor cell of a type selected from the group consisting of breast cancer, ovarian cancer, colon cancer, prostate cancer, liver cancer, lung cancer, gastric cancer, esophageal cancer, urinary bladder cancer, melanoma, leukemia, and lymphoma.

The present invention also provides a method for predicting the likelihood that a cancer patient will respond to therapy with a microtubule-targeting drug comprising obtaining a tumor cell sample from the patient and analyzing DNA in the tumor cell sample to determine the presence or absence of LOH at the M40 β-tubulin gene locus within chromosomal locus 6p25, where LOH does not comprise a mutation in the M40 β-tubulin gene that affects the binding of a microtubule-targeting drug to β-tubulin, and where the presence of LOH is indicative of an increased likelihood that the cancer patient will respond to therapy with the microtubule-targeting drug.

Embodiments of the present disclosure employ, unless otherwise indicated, conventional techniques of synthetic organic chemistry, cell biology, cell culture, biochemistry, molecular biology, transgenic biology, microbiology, recombinant DNA, immunology, and the like, which are within the skill of the art. Such techniques are explained fully in the literature (See, e.g., Molecular Cloning, A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo (Cold Spring Harbor Laboratory Press, Cold Spring Harbor, N.Y., 1986).

The following examples are offered by way of illustration and not by way of limitation.

### EXPERIMENTAL

### Example 1

Acquired tubulin mutations represent the main mechanism by which cancer cells become resistant to drugs that target microtubules (Kavallaris et al. (2001) Cancer Research 61:5803-5809; Giannakakou et al. (1997) J. Biol. Chem. 272:17118-17125; Gonzalez-Garay et al. (1999) J. Biol. Chem. 274:23875-23882; Giannakakou et al. (2000) Proc. Natl. Acad. Sci. USA 97:2904-2909; He et al. (2001) Molecular Cancer Therapeutics 1:3-10; Hua et al. (2001) Cancer Res. 61:7248-7254; Mehdi et al. (2001) Proc. American Assoc. Cancer Res. 42:920). However, the temporal sequence of the molecular events that occur during the development of drug resistance to microtubule-targeting drugs is not known. To investigate the molecular events that occur during the development of drug resistance to microtubule-specific drugs, as well as the adaptive temporal stages in the development of a stable resistance phenotype, a model of epothilone resistance as described by Giannakakou et al. ((2000) Proc. Natl. Acad. Sci. USA 97:2904-2909) was used.

Drug Resistance Model

The model of Giannakakou et al. ((2000) Proc. Natl. Acad. Sci. USA 97:2904-2909) consists of a pair of cell lines: the parental, drug-sensitive human ovarian carcinoma cell line, 1A9, and the epothilone A-resistant clone, 1A9-A8. Previous characterization of 1A9-A8 cells revealed that the epothilone-resistant phenotype is due to an acquired β-tubulin mutation at residue β274 (Thr to Ala) (Giannakakou et al. (2000) Proc. Natl. Acad. Sci. USA 97:2904-2909). Mutation of this residue, located within the taxane-binding pocket on β-tubulin (Nettles et al. (2004) Science 305:866-869; Nogales et al. (1998) Nature 391:199-203) confers a 40-fold resistance to epothilone A (Epo A). In an effort to gain insight into the molecular evolution leading to this 40-fold drug resistance phenotype, an earlier isolate of 1A9-A8 clone, 1A9-A8^{E}, was examined. This 1A9-A8^{E} early-step isolate is a precursor of the 1A9-A8 late-step isolate, as it was only exposed to the selecting agent for six months, while 1A9-A8 cells endured a 15-month selection process. Growth inhibition assays revealed that this early-step isolate was only 10-fold resistant to the selecting agent, epothilone A, unlike the 40-fold resistance displayed by its later-step successor, the 1A9-A8 clone (Table 1).

TABLE 1
CYTOTOXICITY PROFILE OF EPOTHILONE
A RESISTANT CELLS

| | 1A9 | 1A9-A8^{E} | Relative | 1A9-A8 | Relative |
|---|---|---|---|---|---|
| | IC50 | IC50 | Resistance | IC50 | Resistance |
| Epothilone A | 3.2 | 32 | 10 | 125 | 39 |
| Epothilone B | 0.9 | 8 | 9 | 29 | 32 |
| Paclitaxel | 1.5 | 9 | 6 | 15 | 10 |

Cytotoxicity profile of Epothilone A resistant cells to drugs acting on microtubules. The IC50 values, expressed in nM, are obtained following 72 hour exposure to the drug. Relative Resistance is calculated as the ratio of the IC 50 of each respective drug against the resistant clone divided by that obtained against the parental 1A9 cells.

The β-tubulin Gene Status Correlates with Extent of Drug Resistance

To examine whether alterations in the tubulin gene status could account for the differential drug sensitivity displayed by these clones, cDNA from the predominant tubulin isotype (gene M40) from 1A9-A8^{E} cells was sequenced. The results of this analysis clearly demonstrate that both the wild-type (wt) and Mutant ^{Thr}β274^{Ile} tubulin alleles are expressed in the 1A9-A8^{E} cells. In contrast, the 1A9-A8 cells express only the mutant β-tubulin, consistent with previous observations (Giannakakou et al. (2000) Proc. Natl. Acad. Sci. USA 97:2904-2909). Specifically, the 1A9 parental cell line displays wt sequence for the M40 β-tubulin amino acid Thr274 (ACC), while a homozygous point mutation at the β274 residue (ThrACC to IleATC) is seen in the late-step Epo-resistant clone 1A9-A8, and a heterozygous point mutation for the same residue β274 (ThrACC to ThrACC/IleATC) is observed in the early-step Epo-resistant clone 1A9-A8^{E}. Furthermore, the heterozygous tubulin gene status correlates with reduced levels of drug resistance to the microtubule-stabilizing drugs epothilone A, epothilone B, and Taxol® (PTX); while significantly higher-fold resistance values are observed in the 1A9-A8 cells containing only the mutant tubulin gene (Table 1). Thus, intermediate levels of drug resistance were observed with 1A9-A8^{E} cells, as compared with both the 1A9 wt cells and the 1A9-A8 mutant cells.

Impaired Drug-Induced Tubulin Polymerization Correlates with Tubulin Gene Status

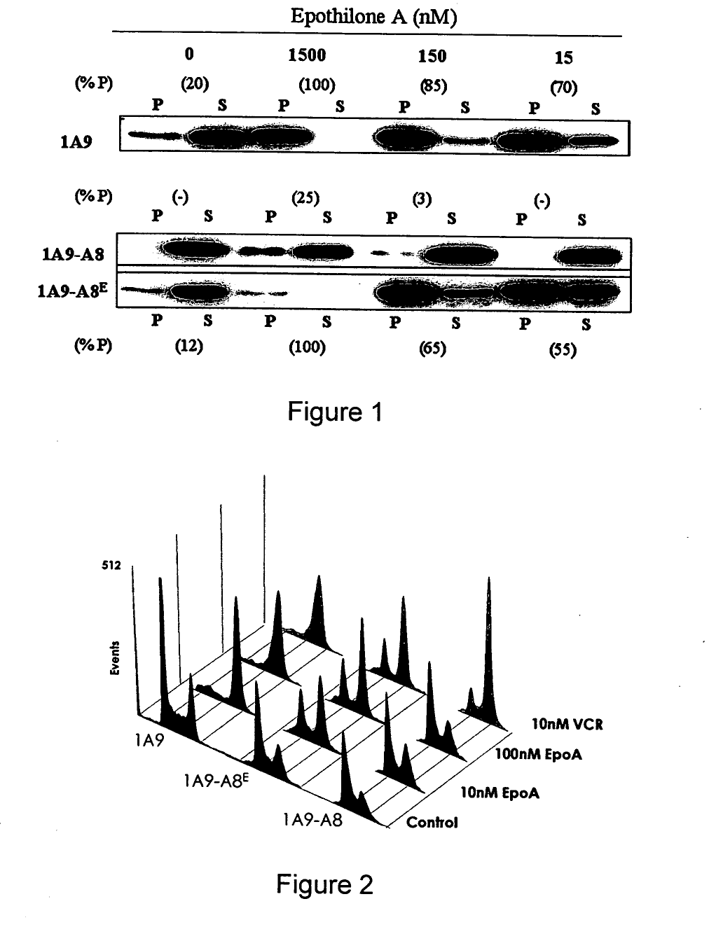

In order to examine whether the tubulin gene status correlates with the ability of epothilone to induce tubulin polymerization in the three related cell lines (1A9, 1A9-A8, and 1A9-A8^{E}), cell-based tubulin polymerization assays were performed (FIG. 1). After treating the cells with escalating doses of Epo A, the cells were harvested in a low salt buffer and then centrifuged to separate the pellet fraction containing the polymerized form of tubulin, from the supernatant that contains the soluble form of tubulin. Under experimental conditions, the untreated controls from all three cell lines contained most of the cellular tubulin in the supernatant fraction, thus in the soluble or unpolymerized form. In the parental cell line (1A9), treatment with Epo A led to a dose-dependent increase in tubulin polymerization, as indicated by the shift of total tubulin from the supernatant to the pellet fractions. In contrast, Epo A had almost no effect on tubulin polymerization in the late-step 1A9-A8 cells, with the majority of the tubulin remaining in the soluble form even at the highest drug concentration (1500 nM), as expected due to the mutant-only tubulin gene status. The intermediate selection step, represented by the 1A9-A8^{E} cells, showed an intermediate degree of tubulin polymerization following drug treatment, consistent with both the wt and the mutant allele being expressed. Treatment with 150 nM of Epo A resulted in 90% of polymerized parental cell tubulin (FIG. 1, top panel), 70% of polymerized tubulin from the early-step 1A9-A8^{E} cells and only 3% of polymerized tubulin from the late-step 1A9-A8 cells (FIG. 1, lower panel). Thus, the effects of Epo A on tubulin polymerization from these three cell lines correlated well with their respective tubulin gene status.

Impaired Drug-Induced G2/M Arrest Correlates with Tubulin Gene Status

Microtubule-targeting drugs are known to induce G2/M arrest as a result of their binding to tubulin or microtubules, blocking cell division at mitosis. Thus, epothilone's ability to induce mitotic arrest in the cell model consisting of isogenic human ovarian cancer cell lines harboring wt, wt/mutant or mutant only β-tubulin genes status was determined.

Epo A treatment resulted in a complete G2/M arrest in the parental 1A9 cells (FIG. 2). As expected, no change was observed in the cell cycle profile of the 1A9-A8 cells upon treatment with Epo A, while a modest G2/M arrest was achieved in the 1A9-A8^{E} clone. Drug treatment with 10 nM of the microtubule-destabilizing drug vincristine, resulted in G2/M arrest in all three cell lines, consistent with the different binding site of this drug on tubulin. Since FACs analysis cannot discriminate between G2 arrest and mitotic arrest, the ability of epothilone to induce mitotic arrest in these cells lines was also tested (Table 2). The results of the mitotic index analysis fully corroborated the cell cycle analysis data as they showed minimal mitotic arrest in the 1A9-A8 clone even at the highest epothilone concentration (100 nM). Collectively, these data reflect the tubulin gene status and the ability of the drug to affect tubulin polymerization (FIG. 1).

TABLE 2
MITOTIC INDEX OF EPOTHILONE A RESISTANT CELLS
Mitotic Index (%)

| Epothilone A (nM) | 0 | 10 | 100 |
|---|---|---|---|
| 1A9 | 6.3 | 82 | 96 |
| 1A9-A8^{E} | 5.2 | 39 | 47 |
| 1A9-A8 | 3.2 | 2.7 | 6.1 |

Mitotic Index of cells treated with the indicated drug concentrations for 24 hr. Approximately 150 cells are scored per drug treatment.

Genomic DNA Sequencing Indicates that wt β-tubulin Gene was Lost in 1A9-A8

As described herein, a tubulin mutation in one of the two alleles is acquired early on during drug selection, while following continuous selection pressure, only the mutant tubulin is expressed. Furthermore, the presence of only mutant tubulin confers higher levels of drug resistance. To examine whether methylation of wt β-tubulin was responsible for the lack of wt β-tubulin expression in 1A9-A8 cells, the 1A9-A8 cells were treated with the DNA demethylating agent 5′-azacytidine. Re-expression of the wt β-tubulin sequence was not detected. The promoter methylation status of β-tubulin was next examined by methylation-specific PCR (Esteller et al. (2001) Hum. Mol. Genet. 10:3001-3007) and found to be unmethylated. To examine whether the gene encoding wt β-tubulin gene was present in 1A9-A8 cells, β-tubulin M40 genomic DNA from the three cell lines was sequenced. The 1A9 cell line displayed a wt β-tubulin sequence, as expected. The 1A9-A8 cells displayed only the mutant ^{Thr}β274^{Ile} sequence, while the intermediate clone 1A9-A8^{E} had both the wild type and mutant sequences. These results suggest that the loss of wt β-tubulin in 1A9-A8 cells is a genetic event.

M40 is Located on 6p25, not on 6p21.33

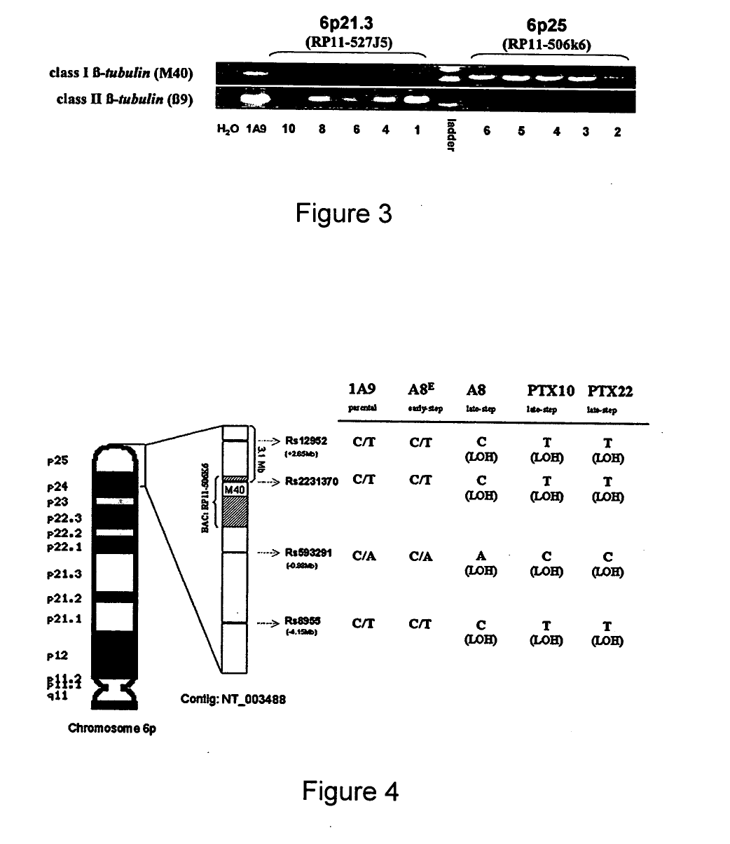

There are seven known isoforms of β-tubulin in the human genome. They share over a 90% nucleotide sequence similarity, with the highest degree of variation being at the C-terminus. Beta-tubulin M40 (also known as class I) is the most predominant of these seven isoforms, accounting for 84.7-98.7% of all expressed β-tubulin in human cancer cells, according to gene expression analysis. Traditional cytogenetic mapping located M40 (gene symbol TUBB) at chromosome 6p21-6pter (Floyd-Smith et al. (1986) Exp. Cell. Res. 163:539-548), and M40 (GeneID 203068) was also placed at 6p21.33 in the Jun. 4, 2004 release of human genome sequence (NCBI, Build 35 version 1). However, a close examination of the NCBI sequence indicated that it corresponds to the β9 tubulin isotype (GeneID 7280), not M40. On the other hand, the cDNA sequence of M40 (AF070561) mapped to 6p25 in an earlier version of the human genome sequence (NCBI, Build 30, Jun. 2002). To resolve this discrepancy, BAC clones for both the 6p21.3 locus (RP11-527J5) and the 6p25 locus (RP11-506K6) were obtained, and genomic PCR primers specific for the M40 and β-9 genes tubulin genes were designed (FIG. 3). The location of each BAC clone was verified by FISH analysis and it was shown that they mapped to their respective loci. Genomic PCR analysis indicated the M40-specific PCR products only amplified from genomic DNA isolated from the RP11-506K6 BAC (located at 6p25), while β-9-specific PCR products only amplified from RP11-527J5 BAC (located at 6p21.3). To further confirm the PCR results, these PCR products were sequenced and their identity validated. Therefore, M40 is located at 6p25, not 6p21.33.

Loss of Heterozygosity for TUBB at 6p25 Results in Increased Taxol® and Epothilone Resistance

To further examine the molecular mechanism leading to loss of wt β-tubulin gene in the late-step 1A9-A8 cells, LOH using single nucleotide polymorphic (SNP) markers was performed. Forty-five SNP markers (see Table 3) from spanning 41.5 mega base pairs along 6p25 were selected to assess the biallelic M40 status of 1A9 parental cells. The heterozygosity status of the 45 selected SNP markers was examined in 1A9 parental cells by PCR amplification of genomic DNA and sequencing. Only four of the 45 tested SNP markers were heterozygous in 1A9 cells; the remaining 41 markers were homozygous (Table 3). These four informative SNP markers were then tested in the early-step 1A9-A8^{E} and late-step 1A9-A8 epothilone-resistant cell lines, as well as in the late-step Taxol®-resistant cells 1A9-PTX10 and 1A9-PTX22 (harboring only mutant β-tubulin alleles at residues β270 and β364, respectively) (Giannakakou et al. (1997) J. Biol. Chem. 272:17118-17125). The results are summarized in FIG. 4. The parental 1A9 and the early-step isolate 1A9-A8^{E} cells contain both alleles, while the late-step isolate clones 1A9-A8, 1A9-PTX10 and 1A9-PTX22 contain only one allele for all 4 SNP markers. All SNPs were located within contig NT_{—}003488, and the deletion encompasses all of the SNP markers in this region. These results indicate that one of the wt TUBB alleles is lost in 1A9-A8, 1A9-PTX10 and 1A9-PTX22 by chromosome loss, consistent with the DNA sequencing analysis.

TABLE 3
HETEROZYGOSITY STATUS OF SNP MARKERS AT 6P25 IN 1A9 PARENTAL
AND TAXOL ® AND EPOTHILONE RESISTANT CELL LINES

| | Distance (Mb) from | Heterozygosity Status | | | | |
|---|---|---|---|---|---|---|
| | start of contig | | 1A9- | 1A9- | 1A9- | 1A9- |
| SNP | NT_003488 | 1A9 | A8^{E} | A8 | PTX10 | PTX22 |
| RS12952 | 0.48 | C/T | C/T | C | T | T |
| RS898768 | 2.77 | Homo | | | | |
| RS1059630 | 2.77 | Homo | | | | |
| RS15286 | 2.82 | Homo | | | | |
| RS6955 | 3.01 | Homo | | | | |
| RS3799212 | 3.12 | Homo | | | | |
| RS2143381 | 3.13 | Homo | | | | |
| RS2143380 | 3.13 | Homo | | | | |
| RS1002852 | 3.13 | Homo | | | | |
| RS2143379 | 3.13 | Homo | | | | |
| RS3799224 | 3.13 | Homo | | | | |
| RS3799225 | 3.13 | Homo | | | | |
| RS727261 | 3.13 | Homo | | | | |
| RS727260 | 3.13 | Homo | | | | |
| RS2231370 | 3.13 | C/T | C/T | C | T | T |
| RS2231371 | 3.14 | Homo | | | | |
| RS1060332 | 3.14 | Homo | | | | |
| RS3088000 | 3.14 | Homo | | | | |
| RS1060334 | 3.14 | Homo | | | | |
| RS3205007 | TUBB | Homo | | | | |
| RS3205008 | TUBB | Homo | | | | |
| RS1054419 | 3.14 | Homo | | | | |
| RS2808001 | 3.14 | Homo | | | | |
| RS2808002 | 3.14 | Homo | | | | |
| RS1054331 | 3.14 | Homo | | | | |
| RS1054310 | 3.14 | Homo | | | | |
| RS1054309 | 3.14 | Homo | | | | |
| RS3209186 | 3.14 | Homo | | | | |
| RS733011 | 3.14 | Homo | | | | |
| RS909961 | 3.14 | Homo | | | | |
| RS3209185 | 3.14 | Homo | | | | |
| RS1054305 | 3.14 | Homo | | | | |
| RS1054304 | 3.14 | Homo | | | | |
| RS1133245 | 3.14 | Homo | | | | |
| RS957638 | 3.14 | Homo | | | | |
| RS3799210 | 3.14 | Homo | | | | |
| RS1054419 | 3.14 | Homo | | | | |
| RS2326177 | 3.15 | Homo | | | | |
| RS3082545 | 3.15 | Homo | | | | |
| RS2808006 | 3.23 | Homo | | | | |
| RS593291 | 4.06 | C/A | C/A | A | C | C |
| RS8980 | 5.08 | Homo | | | | |
| RS9606 | 5.09 | Homo | | | | |
| RS8955 | 7.27 | C/T | C | T | T | T |
| RS13873 | 7.88 | Homo | | | | |
| Total | 45 | 4-Hete | | LOH | LOH | LOH |
| The Heterozygosity status of the 45 selected SNP markers was examined | | | | | | |
| # in 1A9 parental cells by PCR amplification of genomic DNA and sequencing. The | | | | | | |
| # results of this analysis are summarized in this table. The SNP marker reference | | | | | | |
| # number from the NCBI database is shown on the left, the SNP marker location within | | | | | | |
| # the TUBB contig on 6p25 is shown in the middle and the heterozygosity status in 1A9 cells | | | | | | |
| # is shown on the right. Only four from the 45 tested SNP markers were heterozygous | | | | | | |
| # for 1A9 cells. Two SNP markers from within the M40 gene were not heterozygous in 1A9 | | | | | | |
| # cells so they could not be informative in the analysis. | | | | | | |

Fluorescence In Situ Hybridization at Region 6p25

To corroborate the LOH results and to determine whether this LOH event involves the entire chromosome, FISH was performed using the BAC clone RP11-506K6 containing M40 at 6p25 (see scheme in FIG. 4). Before hybridization, sequence analysis confirmed the presence of M40 in this BAC clone. As previously observed, the metaphases of the parental 1A9 and the early-step 1A9-A8^{E} cells showed the presence of two copies of chromosome 6, each displaying BAC hybridization.

In contrast, FISH results from the late-step 1A9-A8 cells, showed the presence of a mixed population. Metaphase spreads from all three cell lines were hybridized with BAC clone RP11-506k6 containing the β-tubulin gene M40, and a centromeric probe for chromosome 6, followed by counterstaining with nucleic acid stain Sytox Blue. The parental 1A9 cells displayed two copies of chromosome 6 as evidenced by the chromosome 6 centromeric probe staining. Both 6 chromosomes displayed staining for the BAC clone indicating two copies of the β-tubulin gene M40. The early-step clone 1A9-A8^{E} presented a similar karyotype as the parental cells, with two copies of chromosome 6 each containing the β-tubulin BAC clone. In the late-step 1A9-A8 cells however, only one chromosome 6 stained for the BAC clone, although both copies of the chromosome 6 were present. In 1A9-A8, the chromosome 6 lost the chromosomal region of 6p25.

In summary, approximately 25% of 1A9-A8 cells displayed a pattern where the BAC probe hybridized to only one copy of chromosome 6, while the second copy of chromosome 6 was devoid of BAC hybridization. In these cells, the LOH event probably involved partial chromosome loss. This result was consistent with the observed loss of heterozygosity for 6p25. The remaining 75% of 1A9-A8 cells exhibited BAC hybridization to both copies of chromosome 6. Based on the LOH analysis showing loss of heterozygosity for 6p25, this result indicated that drug selection of the 1A9-A8 cells lead to the loss of the entire chromosome containing the wt β-tubulin allele followed by duplication of the chromosome containing the mutant β-tubulin allele.

Methods

Cell Lines, Antibodies and Drugs.

The epothilone A resistant cell line, 1A9-A8, was selected from the human ovarian carcinoma 1A9 cells as described by Giannakakou et al. ((2000) Proc. Natl. Acad. Sci. USA 97:2904-2909). The 1A9-A8^{E} clone (expressing both wt and mutant alleles) was an intermediate isolate in the selection process of 1A9-A8 (mutant allele only) cells. These cells were cultured in RPMI 1640 medium (Cellgro, Herndon, Va.) supplemented with 10% fetal bovine serum (Invitrogen, Carlsbad, Calif.) and 1% Penicillin-Streptomycin (Cellgro), and grown as monolayers at 37° C. in a 5% CO₂ tissue culture incubator. The mouse monoclonal anti-α-tubulin (DM1α) antibody used is from Sigma Chemical Co. (St. Louis, Mo.). Both epothilones A and B were a generous gift from the laboratory of K. C. Nicolaou (The Scripps Research Institute, La Jolla, Calif.). Paclitaxel (Taxol®) was purchased from Sigma Chemical Co. and vincristine from Eli-Lilly (Indianapolis, Ind.).

Drug Sensitivity Assay.

Cytotoxicity assays using the protein-staining sulforhodamine B (SRB) method were performed in 96-well plates, as described by Giannakakou et al. ((1997) J. Biol. Chem. 272:17118-17125).

Tubulin Polymerization Assay.

Quantitation of the degree of in vivo tubulin polymerization in response to Epothilone A was performed as described by Giannakakou et al. ((1997) J. Biol. Chem. 272:17118-17125). Briefly, cells were plated in 24-well plates. The following day, they were exposed to increasing concentrations of Epothilone A for a period of 6 hours. Cells were then lysed in a hypotonic buffer [1 mM MgCl₂, 2 mM EGTA, 0.5% Nonidet P-40, 20 mM Tris-HCl, pH 6.8 containing protease inhibitors (Boehringer-Mannheim, Germany)]. The lysed, cells were incubated for 5 minutes at 37° C., and cytoskeletal and cytosolic fractions (containing polymerized (p) and soluble (s) tubulin, respectively) were separated by centrifugation. Equal loading of the fractions was resolved by electrophoresis through 10% SDS polyacrylamide gels, and immunoblotted with an antibody against α-tubulin.

Beta-Tubulin Sequencing.

Total cellular RNA was isolated with the RNeasy Mini Kit (Qiagen, Valencia, Calif.) and the M40 β-tubulin isotype was amplified by RT-PCR using One-Step RT-PCR (Qiagen). Genomic DNA was isolated using the QIAamp DNA Mini Kit (Qiagen). For PCR amplification and sequencing of the M40 β-tubulin isotype, four overlapping sets of primers were used, as summarized in Table 4. The primers were designed to be specific for M40, using GenBank™ accession numbers AP000512 for genomic DNA and AF070600 for cDNA. PCR products were purified using the PCR Purification Kit (Qiagen) and then sent to the sequence core lab of the University of Michigan for DNA sequence analysis.

TABLE 4

PRIMERS USED FOR PCR AMPLIFICATION AND SEQUENC-
ING OF M40 B-TUBULIN

| | | Orien- | |
|---|---|---|---|
| Position | Sequence | tation | Use |
| M40-250 | CTCCGCAAGTTGGCAGTCAAC | Forward | PCR-Tₐ |
| | (SEQ ID NO: 3) | | |
| M40-340 | GGGGATCCATTCCACAAAGTA | Reverse | 58° C. |
| | (SEQ ID NO: 4) | | |
| M40-253 | TTGGCAGTCAACATGGTCC | Forward | Sequenc- |
| | (SEQ ID NO: 5) | | ing |
| M40-324 | CGTTAAGCATCTGCTCATCGACCTCC | Reverse | |
| | (SEQ ID NO: 6) | | |

List of primers used to amplify by PCR and sequence the area around amino acid 274 of the β-tubulin M40 gene.

Cell Cycle Analysis.

Cells were plated in 6-well plates. The following day, they were treated with various concentrations of epothilone A and vincristine for 18 hours. Following treatment, both adherent and floating cells were harvested and pelleted by centrifugation. Cell pellets were suspended in 1 ml of 0.1 mg/ml propidium iodide containing 0.6% NP40 (ICN Pharmaceuticals, Costa Mesa, Calif.) with 1 mg/ml RNase A (Sigma Chemical Co.), then incubated in the dark at room temperature for 30 minutes. Data acquisition and analysis were performed on a FACScan instrument equipped with CellQuest software (Becton Dickinson Immunocytometry Systems, Franklin Lakes, N.J.). Cell cycle analysis was performed with Flowjo (Treestar). All cell cycle experiments were performed at least three times.

Mitotic Index Analysis.

Cells were plated on glass coverslips and treated with drugs for 24 hours. Cells were fixed with ice-cold methanol and DNA was stained with Sytox Green (Molecular Probes, Eugene, Oreg.). Epifluorescence microscopy was used to count a minimum of 500 cells per drug treatment and mitotic figures were scored.

Loss of Heterozygosity Analysis.

Loss of heterozygosity for M40 β-tubulin was examined using PCR primers that amplify single nucleotide polymorphism markers (selected from the human SNP database) around the β-tubulin M40 gene (TUBB) location. A total of 45 SNPs were tested. The PCR products were purified using the PCR Purification Kit (Qiagen) and then sequenced (Sequencing Core, University of Michigan, Ann Arbor, Mich.) to determine if heterozygosity was present. Each PCR reaction was performed at least twice.

Fluorescence In Situ Hybridization Analysis.

The three cell lines (1A9, 1A9-A8 and 1A9-A8^{E}) were induced to be in metaphase by treatment with 0.1 μg/ml colcemid (KaryoMax, Invitrogen) for 4 hours at 37° C. These metaphase cell preparations were harvested and fixed in a 3:1 solution of methanol/acetic acid. One or 2 drops of the cell suspension were added onto each slide and allowed to air-dry. The BAC clone RP11-506k6 (β-tubulin, 6p25: from the RCPI-11 Human BAC Library of the Children's Hospital Oakland Reach Institute BACPAC resources) was labeled by nick translation with digoxigenin-12-dUTP (spectrum-orange, Vysis, Downers Grove, Ill.). Hybridization and immunodetection were performed following the manufacturer's recommendation. For the detection of chromosome 6, a green chromosome 6 centromeric probe (Vysis) was used. Chromosomes were counterstained with Sytox Blue (Molecular Probes) and analyzed by laser scanning confocal microscopy (Zeiss LSM510 axioplasm laser scanning Confocal microscope) using a Zeiss X100 1.3 oil-immersion objective. More than 20 metaphases from each cell line were analyzed.

Discussion

Anticancer drugs select for drug resistance by killing drug-sensitive cells. Taxanes are very effective in the treatment of a wide variety of solid tumors; however, acquired resistance to taxanes limits their clinical efficacy. With continued exposure to the therapeutic drug, a cell develops a mechanism to further increase its chances of survival and expansion. The temporal mechanism by which the 1A9 ovarian carcinoma cells, upon exposure to Epo A, develop moderate drug resistance is described herein and includes a drug binding pocket domain mutation in one allele of the target gene (β-tubulin), and subsequent loss of the chromosomal area around 6p25. This creates a cell type that is now highly resistant to the selecting agent, albeit containing a similar, if not identical, cellular background. The cells with an intermediate level of resistance (1A9-A8^{E}) only have the drug binding pocket domain mutation, and have approximately a ten-fold degree of resistance to the selecting agent, Epo A.

The drug binding pocket domain mutation is located residue β274 (Thr->Ile). This mutation changes the binding pocket and does not allow the drug to bind as efficiently (Giannakakou et al. (2000) Proc. Natl. Acad. Sci. USA 97:2904-2909). Nevertheless, the cell is still producing the wt allele gene and protein, and therefore the drug can bind there and exert its effect. At some point after the acquisition of the β-tubulin mutation, 1A9-A8^{E} cells lose the chromosomal area encompassing 6p25, resulting in the loss of the wt allele and a concomitant higher degree of resistance to Epo A (40 fold).

Losses of heterozygosity are the most common genetic alterations observed in human cancers (Thiagalingam et al. (2001) Proc. Natl. Acad. Sci. USA 98:2698-2702) and are often associated with loss of tumor suppressor genes leading to tumorigenesis. The best known example is LOH of p53. In cancer cells that have lost p53 function, one p53 allele is usually mutated and the other allele is lost due to chromosomal deletion (Baker et al. (1989) Science 244:217-221). However, no studies have correlated the occurrence of LOH with drug resistance. As described herein, a similar event, including mutation of one β-tubulin allele and then loss of the other allele, has been observed during evolution of resistance to Taxol®.

Thus, the taxane-driven selection for mutant tubulin mirrors the process of inactivation of tumor suppressors, consistent with the idea that genetic instability in human cancers is responsible not only for tumorigenesis but also for the development of drug-resistant clones. The model described herein for the development of epothilone resistance in 1A9 cancer cells foresees the acquisition of a β-tubulin mutation in one allele. As the mutation is located within the Taxol®-binding site, epothilone is now unable to bind to some of the M40 β-tubulins. Therefore, the cells are conferred with a moderate degree of resistance to the selecting agent, as long as the other wt allele is still expressed. Upon continued selection with Epo A, the expression of the wt allele disappears, due to loss of the wt β-tubulin allele. Thus, Epo A is unable to effectively bind to any of the M40 β-tubulins, providing the cancer cells with significant growth advantage in the presence of the drug.

Most of late stage 1A9-A8 cells still have two copies of 6p25 even though the LOH analysis suggests that one of the parental alleles is lost. Without being bound by theory, this is likely due to the duplication of the chromosome containing the mutant β-tubulin allele after the loss of the chromosome containing the wt β-tubulin allele. This phenomenon has been frequently observed in association with LOH in human cancers (Thiagalingam et al. (2001) Proc. Natl. Acad. Sci. USA 98:2698-2702).

The results described herein, reveal a new mechanism of taxane resistance that is clinically important given the fact that LOH in chromosome 6p is frequently encountered in human tumors (Arias-Pulido et al. (2004) Genes Chromosomes Cancer 40:277-284; Loeb et al. (2003) Proc. Natl. Acad. Sci. USA 100:776-781; Chatterjee et al. (2001) Cancer Res. 61:2119-2123; McEvoy et al. (2003) Genes Chromosomes Cancer 37:321-325; Miyai et al. (2004) Gynecol. Oncol. 94:115-120; Rodriguez et al. (2005) Cancer Immunol. Immunother. 54:141-148; Hurst et al. (2004) Oncogene 23:2250-2263). In addition, LOH analysis of the 6p25 region in cervical cancer has revealed two as yet unidentified tumor suppressor genes (Chatterjee et al. (2001) Cancer Res. 61:2119-2123). Some of these tumors may lose one copy of 6p during tumorigenesis, leaving them with only one intact copy of the β-tubulin gene. Based on the model described herein, these tumors may have a high likelihood of acquiring a second β-tubulin mutation and become resistant to microtubule-polymerizing agents. This, in conjunction with the unstable human cancer genome that could mutate tubulin in response to treatment with taxanes, provides a rational basis for clinical drug resistance.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for determining microtubule-targeting drug resistance in a cancer patient, said method comprising:
a) obtaining a tumor cell sample from said patient; and
b) analyzing DNA in said tumor cell sample to determine the presence or absence of a loss of heterozygosity at the M40 β-tubulin gene locus within chromosomal locus 6p25, wherein determining said loss of heterozygosity comprises screening for at least one mutation in the M40 β-tubulin gene that affects the binding of a microtubule-targeting drug to βtubulin;
wherein the presence of said loss of heterozygosity is indicative of microtubule-targeting drug resistance in said patient.

2. The method of claim 1, wherein said microtubule-targeting drug is a microtubule-stabilizing drug.

3. The method of claim 2, wherein said microtubule-stabilizing drug is a taxane.

4. The method of claim 3, wherein said taxane is paclitaxel or an analog, derivative, or prodrug thereof.

5. The method of claim 2, wherein the microtubule-stabilizing drug is an epothilone.

6. The method of claim 5, wherein said epothilone is epothilone A, epothilone B, or an analog, derivative, or prodrug thereof.

7. The method of claim 1, wherein said microtubule-targeting drug is a microtubule-destabilizing drug.

8. The method of claim 7, wherein said microtubule-destabilizing drug is vincristine or an analog, derivative, or prodrug thereof.

9. The method of claim 1, wherein said mutation results in an amino acid substitution in said β-tubulin at amino acid positions 26, 172, 198, 231, 240, 270, 274, 282, 292, 350, or 364.

10. The method of claim 9, wherein said amino acid substitution is Asp26Glu, Ser172Ala, Glu198Gly, Ala231Thr, Leu240Ile, Phe270Val, Thr274Ile, Thr274Pro, Arg282Gln, Gln292Glu, Lys350Asn, or Ala364Thr.

11. The method of claim 1, wherein said tumor cell sample comprises tumor cells of a type selected from the group consisting of breast cancer, ovarian cancer, colon cancer, prostate cancer, liver cancer, lung cancer, gastric cancer, esophageal cancer, urinary bladder cancer, melanoma, leukemia, and lymphoma.

12. A method for predicting the likelihood that a cancer patient will respond to therapy with a microtubule-targeting drug, said method comprising:
a) obtaining a tumor cell sample from said patient; and
b) analyzing DNA in said tumor cell sample to determine the presence or absence of a loss of heterozygosity at the M40 β-tubulin gene locus within chromosomal locus 6p25, wherein determining said loss of heterozygosity comprises screening for at least one mutation in the M40 β-tubulin gene that affects the binding of a microtubule-targeting drug to βtubulin;
wherein the presence of said loss of heterozygosity is indicative of a decreased likelihood that said cancer patient will respond to therapy with said microtubule-targeting drug.

13. The method of claim 12, wherein said microtubule-targeting drug is a microtubule-stabilizing drug.

14. The method of claim 13, wherein said microtubule-stabilizing drug is a taxane.

15. The method of claim 14, wherein said taxane is paclitaxel or an analog, derivative, or prodrug thereof.

16. The method of claim 15, wherein the microtubule-stabilizing drug is an epothilone.

17. The method of claim 16, wherein said epothilone is epothilone A, epothilone B, or an analog, derivative, or prodrug thereof.

18. The method of claim 12, wherein said microtubule-targeting drug is a microtubule-destabilizing drug.

19. The method of claim 18, wherein said microtubule-destabilizing drug is vincristine or an analog, derivative, or prodrug thereof.

20. The method of claim 12, wherein said mutation results in an amino acid substitution in said β-tubulin at amino acid positions 26, 172, 198, 231, 240, 270, 274, 282, 292, 350, or 364.

21. The method of claim 20, wherein said amino acid substitution is Asp26Glu, Ser172Ala, Glu198Gly, Ala231Thr, Leu240Ile, Phe270Val, Thr274Ile, Thr274Pro, Arg282Gln, Gln292Glu, Lys350Asn, or Ala364Thr.

22. The method of claim 12, wherein said tumor cell sample comprises tumor cells of a type selected from the group consisting of breast cancer, ovarian cancer, colon cancer, prostate cancer, liver cancer, lung cancer, gastric cancer, esophageal cancer, urinary bladder cancer, melanoma, leukemia, and lymphoma.
